(19) United States
(12) Patent Application Publication
(10) Pub. No.: US 2014/0276196 A1
(43) Pub. Date: Sep. 18, 2014

(54) System and Method for Allergy Testing

(71) Applicant: ALLERGY DEVICES, INC., San Antonio, TX (US)

(72) Inventors: Mark Q. Niederauer, San Antonio, TX (US); Patrick Strauss, San Antonio, TX (US)

(73) Assignee: ALLERGY DEVICES, INC., San Antonio, TX (US)

(21) Appl. No.: 14/208,158

(22) Filed: Mar. 13, 2014

Related U.S. Application Data
(60) Provisional application No. 61/793,863, filed on Mar. 15, 2013.

(51) Int. Cl. A61B 5/00

(52) U.S. Cl. CPC A61B 5/411
USPC 600/556

(57) ABSTRACT

An allergy testing device is formed by a blister pack containing a series of collapsible chambers each associated with an allergen and a lancet. The allergy testing device may be placed adjacent to a patient's skin and actuated by applying pressure to the collapsible chambers to open the chambers and release the reagents contained therein. The lancets deliver the allergen to the skin. The allergy testing device may apply marking to the skin to allow accurate measurement of the allergy testing results.

### CROSS-REFERENCE TO RELATED APPLICATION

This application claims priority to U.S. Provisional Application No. 61/793,863 filed Mar. 15, 2013, the entire contents of which is specifically incorporated by reference herein without disclaimer.

### BACKGROUND OF THE INVENTION

1. Field of the Invention

The present invention relates generally to systems and methods for performing allergy skin tests.

2. Description of Related Art

More than 60 million Americans suffer from allergic diseases, which is the 6th leading cause of chronic disease in the United States. The disease imposes severe restrictions on the physical, psychological and social aspects of patients' lives, which is often under-estimated.

The primary method for testing for allergies in the United States is the skin percutaneous test. A skin test involves a prick with a drop of allergen extract on the surface of the arm or back. This is a well-known technique involving penetration of the allergen into a small area of the skin and then visually observing the reaction, if any, of the skin to the allergen. So far, skin tests have required a complex arrangement of multiple components often coming from different sources stored in different areas of a clinic depending in part on climate control. These typically include: a tray, a well holder assembly, a well holder assembly lid, a skin prick device with multi-headed pointed ends and biologic allergen extracts in vials to be poured into the wells, a pen or marker and a separate measuring device such as a ruler.

To perform a skin test, there are multiple steps. First, the trays must be labeled. Second, individual extracts corresponding to the labels on the trays must be poured into the reservoir wells in the well-holder assembly. Both the extracts and the assembly are stored in a refrigerator. These steps are typically only done once for every 40 patients or so. Following these steps, the person does the following for each patient tested. First, the patient's back or skin is marked in a predetermined sequence, usually A, B, C and so on. Each letter corresponds to the label contained on the trays or otherwise to identify the allergens used for the test. Each label contains an array or battery of typically 8 to 10 allergens. After the patient is labeled, the technician typically removes the skin-test device from its packaging and places the device prongs in the wells to adhere a small amount of test solution to the sharp pointed ends from each test-solution well. The device is removed from the well and applied to a site on the patient's skin in a rocking fashion in accordance with the predetermined sequence corresponding to the labels. The person conducting the test then reviews the reaction on the skin and records the results typically using a hand-held measuring device such as a ruler.

These steps are time-consuming and wasteful of biologic allergen extracts. Furthermore, there is a considerable chance of operator error, including spillage, inaccurate marking, cross-contamination, and inaccurate reading. Thus, there is a need for a simple, accurate and reliable system for conducting allergy skin tests.

### SUMMARY OF THE DISCLOSURE

In general, embodiments of the present disclosure provide new and improved systems, methods and devices for allergy testing that overcome one or more of the problems with existing systems.

According to one aspect, an allergy testing device comprises an allergen layer having a first surface and a second surface, at least one allergen chamber on the allergen layer, a delivery layer coupled to the second surface of the allergen layer, and at least one lancet coupled to the delivery layer. At least one lancet is associated with the at least one allergen chamber and has a channel extending therethrough. The channel may be adapted to transport fluid from a first end to a second end of the lancet.

An actuation layer which is adapted to open the allergen chambers may be coupled to the top surface of the allergen layer. The actuation layer may be mechanically activated, and the mechanical activation may comprise applying pressure.

The actuation layer may comprise a plurality of piercing elements facing the allergen layer. The piercing elements may be disposed on one side of the actuation layer, and the actuation layer may be reversible so that the piercing elements may face away from the allergen layer.

A cover for covering the actuation layer may be provided.

A marking layer may be coupled to the allergen layer for marking a patient's skin with identifying information. The marking layer may comprise means for temporarily marking the patient's skin, such as ink or an adhesive decal.

An adhesive layer for affixing the allergy testing device to a patient's skin may be provided, and a removable protective layer for protecting the adhesive layer may be provided.

A mechanical actuator for opening the at least one allergen chamber may be provided.

According to another aspect, an allergy testing device has a body having a plurality of reservoirs adapted to be filled with allergens, the body having a bottom, patient-facing surface and a top surface. A plurality of applicators are associated with the plurality of reservoirs, and each applicator is disposed within an associated reservoir and is adapted to, upon receiving pressure, pierce the associated reservoir to release the antigen contained in the reservoir. The body may comprise a first membrane sealed to a second membrane to form the plurality of reservoirs. The allergen may be a liquid or may be dry.

The applicators may be adapted to deliver the antigen in the associated reservoir to a precise area of a patient's skin, and the applicators may comprise lancets adapted to abrade a patient's skin.

A marking layer may be provided on the patient-facing surface of the body for marking a patient's skin with identifying information. The marking layer may be ink or an adhesive decal. A removable protective layer for protecting the marking layer prior to use may be provided.

In accordance with another aspect, a testing device comprises a delivery layer comprising a plurality of lancets, each lancet having a channel extending between a first end and a second end of the lancet, The delivery layer is adapted to be placed adjacent to a patient's skin so that the second end of the lancets contact the patient's skin. An allergen layer is coupled to the delivery layer and comprises a plurality of chambers adapted to be filled with an allergen. Each chamber is associated with the first end of a lancet, wherein, when a chamber is opened, the antigen contained therein is transported from the first end of the associated lancet to the second end of the lancet through the channel.

The second end of the lancet may be adapted to abrade a patient's skin, and the channel may be adapted to wick liquid from the first end to the second end.

An actuation layer adapted to open the plurality of chambers may be provided, and biasing elements for urging the actuation layer away from the allergen layer may be provided. The actuation layer may comprise a plurality of piercing elements associated with the plurality of chambers, the piercing elements adapted to open the chambers when pressure is applied. The actuation layer may comprise a first side and a second side opposite the first side, and the piercing elements may be located on the first side and the second side may be substantially smooth. The actuation layer may be removable and reversible so that either the first side or second side is adjacent the allergen layer. A cover for covering the actuation layer may be provided. The actuation layer cover may be configured and used to discharge the piercing elements of the actuation layer.

A marking layer may be disposed on the abrasion layer for marking a patient's skin with identifying information. The marking layer may comprise ink or an adhesive decal.

An adhesive layer may be coupled to the allergen layer for adhering the allergy testing device to a patient's skin. A removable protective layer for covering the adhesive layer may be provided.

In accordance with yet another aspect, a method of allergy testing, comprises placing an allergy testing device according to any one of the preceding claims onto a patient's skin and actuating the allergy testing device to deliver allergens to the patient's skin.

The term “coupled” is defined as connected, although not necessarily directly. The terms “a” and “an” are defined as one or more unless this disclosure explicitly requires otherwise.

The terms “substantially,” “approximately,” and “about” are defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, or a component of a system, that “comprises,” “has,” “includes” or “contains” one or more elements or features possesses those one or more elements or features, but is not limited to possessing only those elements or features. Likewise, a method that “comprises,” “has,” “includes” or “contains” one or more steps possesses those one or more steps, but is not limited to possessing only those one or more steps. Additionally, terms such as “first” and “second” are used only to differentiate structures or features, and not to limit the different structures or features to a particular order.

A device, system, or component of either that is configured in a certain way is configured in at least that way, but it can also be configured in other ways than those specifically described.

Any embodiment of any of the systems and methods can consist of or consist essentially of—rather than comprise/include/contain/have—any of the described elements, features, and/or steps. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 is a top perspective view of an allergy testing device in accordance with an exemplary embodiment;

FIG. 2 is a sectional view of the testing-device of FIG. 1;

FIG. 3 is a bottom perspective view of the allergy testing device of FIG. 1;

FIG. 4 is a side view of a lancet according to an exemplary embodiment;

FIG. 5 is a side view of the lancet of FIG. 4 inside an allergen chamber, prior to actuation;

FIG. 6 is a side view of the lancet of FIG. 4 inside an allergen chamber, after actuation;

FIG. 7 is a side view of an actuation device according to an exemplary embodiment;

FIG. 8 is a partial side view of the actuation device of FIG. 7 in combination with an allergy testing device;

FIG. 9 is a top view of a temporary skin decal according to an exemplary embodiment;

FIG. 10 is a side view of the temporary skin decal of FIG. 9 attached to an allergy testing device;

FIG. 11 is an enlarged top view of a portion of the temporary skin decal of FIG. 9 showing the antigen identification label and calibration to measure wheal and flare on skin;

FIG. 12 illustrates an array of decals on a patient;

FIG. 13 is an exploded view of the layers of an allergy testing device according to an exemplary embodiment;

FIG. 14 is a top view of the actuation layer of the allergy testing device of FIG. 13;

FIG. 15 is a side view of the actuation layer of the allergy testing device of FIG. 14;

FIG. 16 is a top view of the delivery layer of the allergy testing device of FIG. 13;

FIG. 17 is a side view of the delivery layer of the allergy testing device of FIG. 13; and

FIG. 18 is a partial bottom view of the delivery layer of the allergy testing device of FIG. 13.

### DESCRIPTION OF ILLUSTRATIVE EMBODIMENTS

Illustrative embodiments of the present disclosure provide systems, devices and methods for allergy skin testing. Referring to FIGS. 1-6, a test device 100 in accordance with an illustrative embodiment comprises a body 102 having a plurality of chambers 104. Each chamber 104 contains an allergen 106 and a piercing element 108.

Body 102 has a top surface 110 and a bottom surface 112. Body 102 is preferably flexible so that it may conform with the shape of a patient's body when applied to the body. Chambers 104 may be disposed on top surface 110, bottom surface 112, or in the middle of body 102. Chambers 104 may be formed separately or integrally with body 102.

In one embodiment, body 102 comprises a “blister pack,” such as those used for unit dose packaging of pharmaceutical tablets and chambers. Blister packs are typically formed of a blister card with a plurality of collapsible chambers covered by a thin foil material. The blister card may be formed by thermoforming a plastic material, such as PVC, coldforming a cold forming foil (CFF) laminate, or any other known technique. After filling the collapsible chambers with allergens and piercing elements, the thin, foil layer is coupled to the blister card to form sealed chambers 104. The material of body 102 is selected to protect allergen 106 from the environment (e.g., light, heat and moisture) to avoid contamination and minimize degradation of allergen 106 prior to use. It should be noted that test device 100 may be used to deliver any substance to the skin, and allergen should be interpreted broadly to include any substance used in test device 100.

Chambers 104 are substantially sealed prior to use to retain allergen 106 and piercing element 108 therein. In one embodiment, each of chambers 104 in test device 100 contains a different allergen 106. For example, chambers 104 in a single test device 100 may contain a set of common environmental allergens or a set of common food allergens which are typically all tested for at once. Chambers 104 may also contain positive and negative controls (usually histamine and saline, respectively) to ensure the validity of any testing. Only a single unit dose of allergen is placed into the chamber 204, thereby improving accuracy and repeatability while minimizing waste and usage as compared to conventional allergy testing methods. Typically, allergen 106 is a liquid. However, allergen 106 may also be a dry or substantially dry coating on piercing element 108. For example, a piercing element may be dipped in an allergen and dried to coat the piercing element with an allergen.

One or more of chambers 104 in test device 100 may be filled with the same allergen. For example, substantially all of the chambers 104 in a particular test device 100 may be filled with the same allergen. Body 102 may be perforated or pre-scored so that individual chambers 104 may be removed from body 102 for individual use. This configuration allows a user to detach and use a single chamber 104, which may be useful for testing for individual allergens which do not form part of a common testing protocol.

In the illustrated embodiment, eight chambers 104 are arranged in a two by four array. The size and shape of body 102 and the number, size and spatial configuration of chambers 104 may vary according to the desired application. It should be noted that storage of test device 100 will take much less room than the multi piece tray, assembly, extract, and device currently used in percutaneous diagnostics. Instead, test devices 102 will be stored as flat sleeves of blister packs taking significantly less space.

Top surface 110 may have indicator 114 to aid a user. For example, a directional arrow 116 may be provided to indicate the proper orientation of testing device 100. Bottom surface 112 is preferably generally flat so that it will conform to a patient's skin. An adhesive 118 may be applied to bottom surface 112 to aid usage by allowing a user to adhere the test device 100 to a patient prior to actuation. A removable protective cover 120 may be provided to protect the adhesive 118 prior to usage. In one embodiment, cover 120 is a protective film.

Piercing element 108 has a first end 122 and a second end 124. First end 122 of piercing element 108 is configured to allow a user to apply force, either directly or indirectly (such as by using a mechanical applicator), to piercing element 108. In one embodiment, first end 122 is configured as a broad pushing surface which is substantially the same size as a chamber 104 to allow a user to easily push the piercing element. This configuration also helps to maintain the correct orientation of piercing element 108 in chamber 104. Second end 124 is configured to open chamber 104 to dispense allergen 106. Second end 124 may be pointed or otherwise configured to abrade a patient's skin and assist delivery of allergen 106 to the epidermis. Chamber 104 may be configured to retain piercing element 108 in the proper orientation.

Referring to FIG. 6, in operation, test device 100 is placed adjacent to a patient's skin 126. Force F is applied to a chamber 104 to collapse the chamber and force the piercing element 106 through the opposite side of chamber 104, thereby opening chamber 104 and allowing allergen 106 to be delivered to a patient's skin. Second end 124 of piercing element 108 may be configured to abrade or prick the patient's skin to deliver allergen 106 to the skin and test for an allergic reaction. Preferably, piercing element 108 only abrades the epidermis. After all of piercing elements 108 in test device 100 are actuated, test device 100 is removed, and, after a sufficient amount of time has passed, the test results are read. The test device 100 is intended to be a single use device which is discarded after use.

Referring to FIGS. 7 and 8, a mechanical actuator 130 may be provided to apply and actuate test device 100. Actuator 130 comprises a handle 132 and one or more arms 134 extending from handle 132 to a rocker 136. Arms 134 may be attached to a rotating axle 138 to allow handle 132 to pivot with respect to rocker 136. Test device 100 may be releasably attached to a bottom surface 140 of rocker 136. For example, test device 100 may be attached to actuator 130 with a low-tack adhesive or a releasable mechanical attachment. In use, test device 100 is attached to bottom surface 140 of rocker 136, and then pressed against a patient's skin 142. Pressure is applied via handle 132 to actuate piercing elements 108, open the chambers 104, and prick the patient's skin 142. Mechanical actuator 130 may be a single use device or may be reusable.

Referring to FIGS. 9-11, a marking layer, such as decal 150 may be removably coupled to bottom surface 112 of test device 100. Decal 150 is a thin flexible membrane, and a temporary adhesive layer 154 may be provided on a patient facing surface 158 of the decal. A removable protective cover 156 may be provided to protect adhesive layer 154 prior to use. In operation, protective cover 156 is removed to expose adhesive layer 154. Test device 100 is pressed against a patient's skin and actuated (either manually or with an actuator device) to deliver allergens to the patient's skin. Test device 100 is removed, while decal 150 is decoupled from test device 100 and adheres to the patient's skin to hold decal 150 in place during testing.

Openings 152 in the decal are provided to allow application of allergen to a patient's skin while diminishing the risk of cross-contamination between testing sites. Labels 160 indicate the type of allergens, and a calibrated scale 162 is provided to assist a user with accurately reading test results. Practice guidelines suggest that skin test results include the diameter of the wheal and flare, usually measured in millimeters. Once testing is complete, decal 150 may be removed from the patient and discarded.

Other means of temporarily marking a patient may also be provided. For example, a temporary tattoo or temporary ink may be provided on the bottom surface 112 of test device 100 to temporarily mark a patient and label the test sites. Accurately labeling the test sites minimizes erroneous test readings.

More than one test device 100 may be utilized at the same time. FIG. 12 illustrates the application of nine testing devices in a three by three array on a patient's back. In this example, each test device 100 contains a different set of allergens.

Referring to FIGS. 13-18, a test device 200 in accordance with an illustrative embodiment comprises an allergen layer 202 with a top surface 204 and an opposing bottom surface 206. Allergen layer 202 includes a plurality of allergen chambers 208 for holding allergen, and is constructed similarly to previously described body 102. Allergen chambers 208 may be opened to release allergen. Allergen layer 202 is coupled to a delivery layer 210. Delivery layer 210 has a plurality of lancets 212 which correspond to the plurality of allergen chambers 208. Lancets 212 are configured to deliver allergen from the allergen chamber to a patient's skin. In one embodiment, lancets 212 have a first end 214 which are configured to be disposed near an allergen chamber 208 and a second end 216 configured to reach a patient's skin. An opening or channel extends therethrough to allow allergen to pass through the delivery layer. The channel may be configured to use wicking, or capillary action, to transport allergen from first end 214 to second end 216. First end 214 may be configured to aid in delivering allergen from an allergen chamber 208 to an associated lancet 212. In one particular embodiment, first end 214 is funnel shaped. Delivery layer 210 may be formed of a semi-rigid plastic material, and may have a rim 220 around the outer perimeter. Allergen layer 202 is sized to fit inside the rim 220 and may be held in place by mechanical or adhesive means.

An actuation layer 222 has a top surface 224 and an opposing bottom surface 226. The bottom surface 226 has a plurality of piercing elements 228 which are positioned to coincide with allergen chambers 208. Biasing members 230 are provided to urge actuation layer 222 away from allergen layer 202 prior to actuation. In the illustrated embodiment, biasing members 230 comprise four leaf springs, although different types and orientations of springs or other mechanical items may be used. Actuation layer 222 is sufficiently rigid so that it may be pressed toward the allergen layer to open allergen chambers 208 with piercing elements 228. Piercing elements 228 may be sharp to facilitate opening allergen chambers 208, and may have a complementary shape to the first ends 214 of lancets 212. This allows the piercing elements to puncture the top and bottom of the allergen chambers 208 and deliver allergens to the lancets 212. The actuation layer may be removable and reversible so that the piercing elements face away from the allergen layer to prevent inadvertent actuation.

A marking layer 232 may be provided on the bottom surface 206 of the delivery layer 210. Marking layer 232 may be directly applied to the bottom surface (e.g., it may be a transferrable ink on bottom surface 206). Alternatively, marking layer 232 may be a separate layer attached to bottom surface 206 by a mechanical attachment, such as pins 234 which engage openings (not shown) in marking layer 226. Other methods, such as adhesives or frictional engagement with peripheral rim 220 of delivery layer 210, may also be used. Marking layer 226 may be configured as a decal or may utilize ink or a tattoo as previously described.

Top and bottom covers 236, 238 may be provided to protect test device 200 prior to use. Peripheral rims 240, 242 which are adapted to removably engage rim 220 of delivery layer 210 may be provided on top and bottom covers 236, 238. For example, rims 220, 240, and 242 may have matching ridges and grooves to frictionally engage one another.

To use test device 200, top and bottom covers 236, 238 are removed. The bottom surface of test device 200 is placed at a desired location on a patient's body, and actuation layer 222 is pressed firmly toward the patient. Piercing elements 228 open allergen chambers 208 and allow allergen to enter into openings 218 in lancets 212. The allergens pass through the lancets and are delivered to the patient's skin. The capillary action of openings 218 delivers a precise amount of allergen. The marking layer 226 adheres to the patient's skin (if configured as a decal) or otherwise marks the patient's skin (if configured as a tattoo or ink). Device 200 is removed and discarded. After a sufficient period of time has passed, the test results are read using the indicia left by the marking layer. The tissue reaction or test results may also be photographed and automatically read using digital tools and image analysis applications. After completion, the markings are removed by peeling off the decal or washing off any ink, and testing is complete.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. For example, components may be combined as a unitary structure, and/or connections may be substituted (e.g., threads may be substituted with press-fittings or welds). Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An allergy testing device, comprising:
an allergen layer having a first surface and a second surface;
at least one allergen chamber on the allergen layer containing an allergen;
a delivery layer coupled to the second surface of the allergen layer; and
at least one lancet coupled to the delivery layer, wherein the at least one lancet is associated with the at least one allergen chamber, and the at least one lancet has a channel extending therethrough.

2. The allergy testing device according to claim 1, wherein the channel is adapted to transport fluid from a first end to a second end of the lancet.

3. The allergy testing device according to claim 1, further comprising an actuation layer coupled to the top surface of the allergen layer, the actuation layer being adapted to open the allergen chambers.

4. The allergy testing device according to claim 1, wherein the actuation layer is mechanically activated.

5. The allergy testing device according to claim 4, wherein the mechanical activation comprises applying pressure.

6. The allergy testing device according to claim 3, wherein the actuation layer comprises a plurality of piercing elements facing the allergen layer.

7. The allergy testing device according to claim 6, wherein the piercing elements are disposed on one side of the actuation layer, and the actuation layer is reversible so that the piercing elements may face away from the allergen layer.

8. The allergy testing device according to claim 3, further comprising a cover for covering the actuation layer.

9. The allergy testing device according to claim 3, further comprising a marking layer coupled to the allergen layer for marking a patient's skin with identifying information.

10. The allergy testing device according to claim 9, wherein the marking layer comprises means for temporarily marking the patient's skin.

11. The allergy testing device according to claim 9, wherein the marking layer comprises ink on the allergen layer.

12. The allergy testing device according to claim 9, wherein the marking layer comprises an adhesive decal.

13. The allergy testing device according to claim 9, further comprising an adhesive layer for affixing the allergy testing device to a patient's skin.

14. The allergy testing device according to claim 13, further comprising a removable protective layer for protecting the adhesive layer.

15. The allergy testing device according to claim 1, further comprising a mechanical actuator for opening the at least one allergen chamber.

16.-39. (canceled)
